(19) United States
(12) Patent Application Publication
(10) Pub. No.: US 2024/0099617 A1
(43) Pub. Date: Mar. 28, 2024

(54) DIFFUSE OPTICAL IMAGING/TOMOGRAPHY USING META-OPTICS

(71) Applicant: Applied Materials, Inc., Santa Clara, CA (US)

(72) Inventors: David Alexander SELL, Santa Clara, CA (US); Paul GALLAGHER, Santa Clara, CA (US); Christopher G. TALBOT, Emerald Hills, CA (US); Christopher John WRIGHT, London (GB); Harry Michael CRONIN, Cambridge (GB)

(21) Appl. No.: 18/477,444

(22) Filed: Sep. 28, 2023

Related U.S. Application Data
(60) Provisional application No. 63/411,043, filed on Sep. 28, 2022.

(51) Int. Cl. A61B 5/1455; A61B 5/00; A61B 5/024; G01N 21/47; G01N 33/02

(52) U.S. Cl. CPC A61B 5/14552; A61B 5/0073; A61B 5/02427; A61B 5/4809; G01N 21/4738; G01N 21/4795; G01N 33/025

(57) ABSTRACT

Method and apparatuses for diffuse optical tomography (DOT) are disclosed herein. A DOT device includes a substrate, one or more radiation sources, a plurality of detectors, and structures disposed over the second surface of the plurality of detectors. The one or more radiation sources are disposed over or under a surface of the substrate. Each detector of the plurality of detectors has a first surface and a second surface. The first surface is opposite the second surface. The first surface of the plurality of detectors disposed over or under the surface of the substrate. The method of DOT method of includes emitting and scattering radiation from one or more sources of a DOT device; detecting scattered radiation with a plurality of detectors of the DOT device; and translating the scattered radiation that is detected into data.

### CROSS-REFERENCE TO RELATED APPLICATIONS

This application claims benefit of U.S. provisional patent application Ser. No. 63/411,043, filed Sep. 28, 2022, which is herein incorporated by reference.

### BACKGROUND

### Field

Embodiments described herein generally relate to optical tomography devices.

### Description of the Related Art

Diffuse optical tomography (DOT) is a non-invasive imaging technique that uses radiation, such as near infrared (NIR) radiation or visible light. DOT allows for imaging of biological tissue to provide functional and anatomical information. For example, it is desirable to differentiate between oxygenated and deoxygenated hemoglobin to identify tissue. Due to the different absorption spectra of the oxygenated and deoxygenated hemoglobin, spectroscopic separation of the materials using the scattered radiation is enabled. Generated radiation is propagated through tissue, which is the diffusive media. A computer is used to control the radiation illumination and detection in and out of the diffusive media. The detected signal is then used to solve the inverse scattering problem and extract data and images of structures in the diffusive media.

The radiation sources and detectors in current DOT systems may be fiber-coupled LEDs and photodiodes. The current DOT systems are slow, difficult to use, not portable, and have poor image quality. Therefore, there is a need for an improved optical tomography device and a method of performing optical tomography.

### SUMMARY

Embodiments described herein generally relate to optical tomography devices.

In one embodiment, a diffuse optical tomography (DOT) device is disclosed. The DOT device includes a substrate, one or more radiation sources, a plurality of detectors, and structures disposed over the second surface of the plurality of detectors. The one or more radiation sources are disposed over or under a surface of the substrate. Each detector of the plurality of detectors has a first surface and a second surface. The first surface is opposite the second surface. The first surface of the plurality of detectors disposed over or under the surface of the substrate. The structures cause diffraction, refraction, or filtering of the radiation entering the detectors.

In another embodiment, a diffuse optical tomography (DOT) device is disclosed. The DOT device includes a source substrate, a detector substrate, one or more sources disposed over or under a source surface of the source substrate, a plurality of detectors, and a plurality of structures. Each detector of the plurality of detectors has a first surface and a second surface. The first surface opposite the second surface. The first surface of the plurality of detectors disposed over or under a detector surface of the detector substrate. The plurality of structures are disposed over the second surface the plurality of detectors. The structures cause diffraction, refraction, or filtering of the radiation entering the detectors.

In yet another embodiment, a method is disclosed. The method includes emitting radiation from one or more sources of a DOT device, wherein the radiation is scattered; detecting scattered radiation with a plurality of detectors of the DOT device, wherein the plurality of detectors have a plurality of structures disposed thereover; and translating the scattered radiation that is detected into data. The structures cause diffraction, refraction, or filtering of the radiation entering the detectors.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only typical embodiments of this disclosure and are therefore not to be considered limiting of its scope, for the disclosure include other useful and effective embodiments.

FIG. 1A illustrates a cross-sectional view of the DOT device with an interspersed array arrangement of radiation sources and detectors, according to embodiments described herein.

FIG. 1B illustrates a view of a surface of a substrate of the DOT device having a rectangular surface, according to embodiments described herein.

FIG. 1C illustrates a view of a surface of a substrate of the DOT device having to an annular configuration, according to embodiments described herein.

FIG. 1D illustrates a cross-sectional view of the DOT device with separate detector and source (SDS) array arrangement, according to embodiments described herein.

FIG. 1E illustrates a view of a surface of a DOT device with a latticed array arrangement, according to embodiments described herein.

FIG. 1F illustrates a view of a structure of a first configuration, according to embodiments described herein.

FIG. 1G illustrates a view of a structure of a second configuration, according to embodiments described herein.

FIG. 2 is a flow diagram of a method of diffuse optical tomography, according to embodiments described herein.

FIG. 3A illustrates a schematic cross-sectional view of a DOT device with angularly selective structures, according to embodiments described herein.

FIG. 3B illustrates a schematic cross-sectional view of an array of detectors with a range of angularly selective structures, according to embodiments described herein.

FIG. 3C illustrates a schematic cross-sectional view of multiple arrays of a detectors with repeating groups of angularly selective structures, according to embodiments.

FIGS. 4-9 illustrate exemplary uses of a DOT device, according to embodiments described herein.

### DETAILED DESCRIPTION

Embodiments described herein generally relate to optical tomography devices.

Described herein are DOT devices and methods of utilizing DOT devices for maximizing image quality, increasing information content (e.g., blood, blood oxygen, etc.), reducing acquisition time, and minimizing computational intensity, cost, and DOT device size.

Diffuse optical tomography (DOT) is a non-invasive imaging technique that typically uses radiation, such as near infrared (NIR) radiation or visible light. The radiation has wavelengths of 500 nm to 2500 nm. More particularly, 600 nm to 1200 nm, or 650 nm to 1000 nm. Different ranges may be utilized for different implementations. For example, biological tissue is relatively transparent to radiation in the 700 nm to 900 nm (e.g., near infrared (NIR) radiation) range. In one instance, a range of 650 nm to 750 nm may be utilized to differentiate oxygenated and deoxygenated blood. In another embodiment, a range of 900 nm to 950 nm may be utilized for detecting lipids. In yet another embodiment, a range of 950 nm to 1000 nm may be utilized to detect water. In a DOT device utilizing LED sources and photodiodes optionally connected via fiber cables, the DOT device may be very large, relative to the form factors being targeted, to accommodate all of the fiber cables. One approach to increasing the performance of a DOT device requires increasing the number of sources or detectors in the DOT device. However, the increased number of sources or detectors also requires an increase in fiber cables, computational time, and computing power. Thus, there is a need for an improved device and method of increasing image quality in DOT devices without increasing size, computational time, or required computing power of the device.

Structures are designed to selectively filter by angle, wavelength, polarization, or other qualities of emitted or detected radiation. The structures cause diffraction, refraction, or filtering of the radiation entering the detector. The structures may be configured to be wavelength filters, angular filters, flat lenses, or other types of filters and may perform functions such as edge detection or shape detection. The structures can include nanoantennas, nanopillars, nanorods, nanoslitsn, nanoholes, nanowires, nanodisks, nanoislands, or the like extending from or inside the surface of a substrate and may form metalenses, diffractive gratings, or diffractive lenses. In some embodiments, the metalenses are flat lenses. Metalenses, diffractive gratings, or diffractive lenses may be known as metasurfaces. The structures have a height that is related to the wavelength of the radiation emitted. In some embodiments, the height structures may be from about ⅓ of the wavelength emitted to about 3 times the wavelength emitted. In embodiments in which the substrate is transparent, the DOT device 100 may include sources 102 and detectors 104 disposed over of on either side of the substrate. In embodiments in which the substrate is transparent, the structures are disposed over of on either side of the substrate.

The application of structures to some or all detectors and/or some or all sensors generally reduces the amount of detected radiation in a DOT device. However, the structures reduce the amount of noise (i.e., signal with no information content), thus increasing the information content and improving the signal to noise ratio. With an improved signal to noise ratio, the computational burden on the DOT device is reduced. With the reduction of the computational burden, the DOT device is capable of decreasing the noise in resulting output data and images. Further, the miniaturization and optical functions implemented using the structures may assist in reducing the number of sources and detectors required, fiber cables, computational time, and computing power requirements of the DOT device.

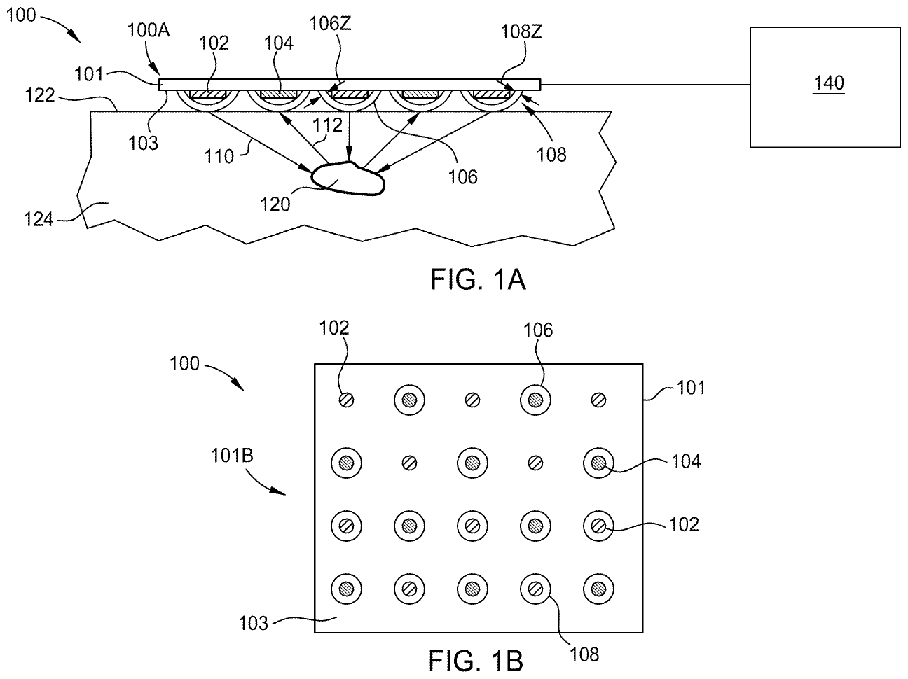

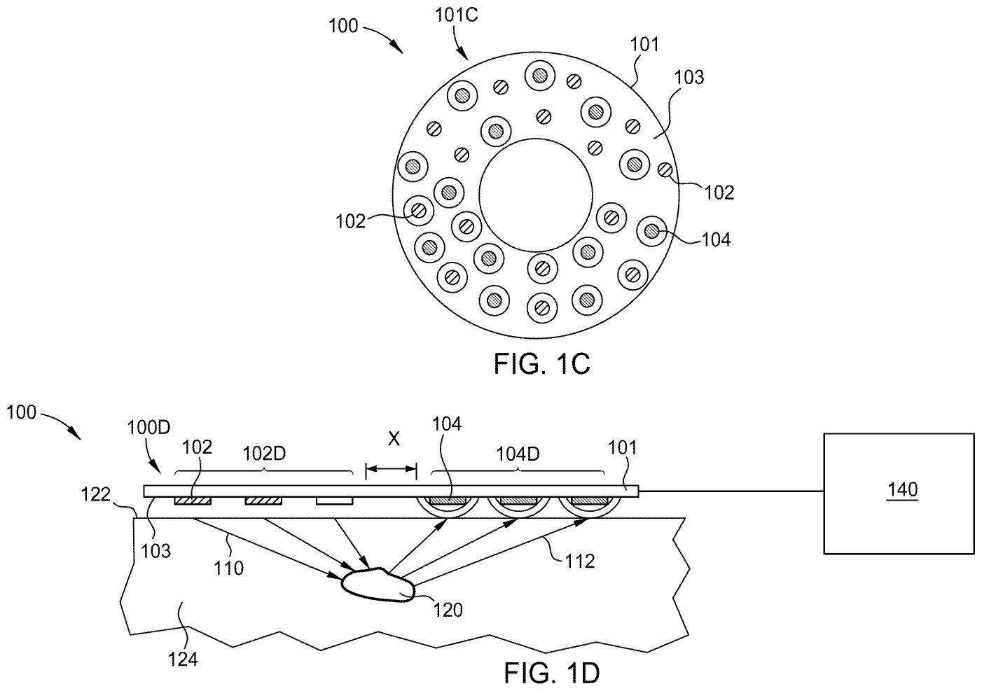

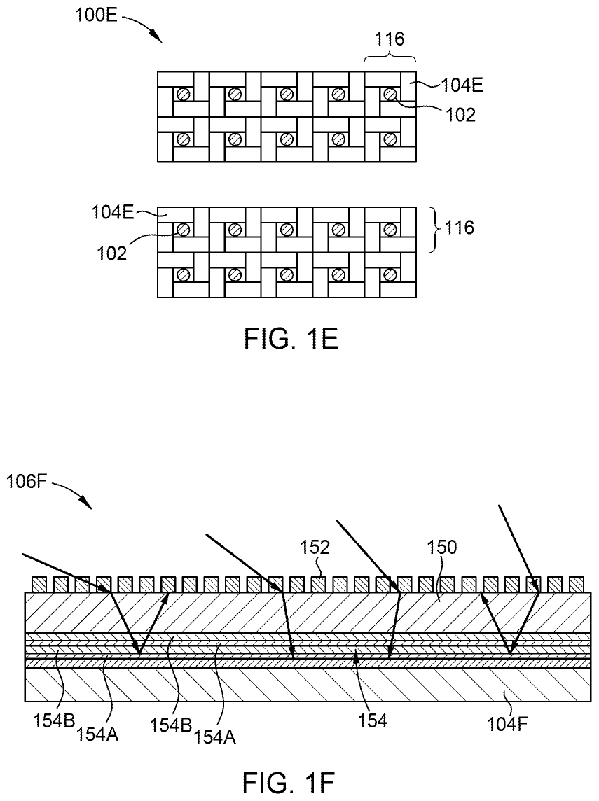

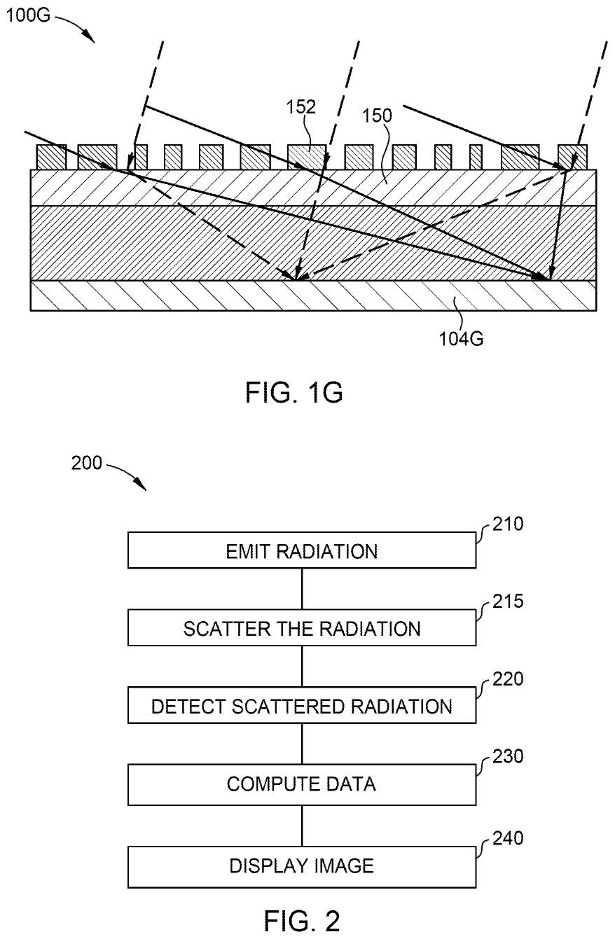

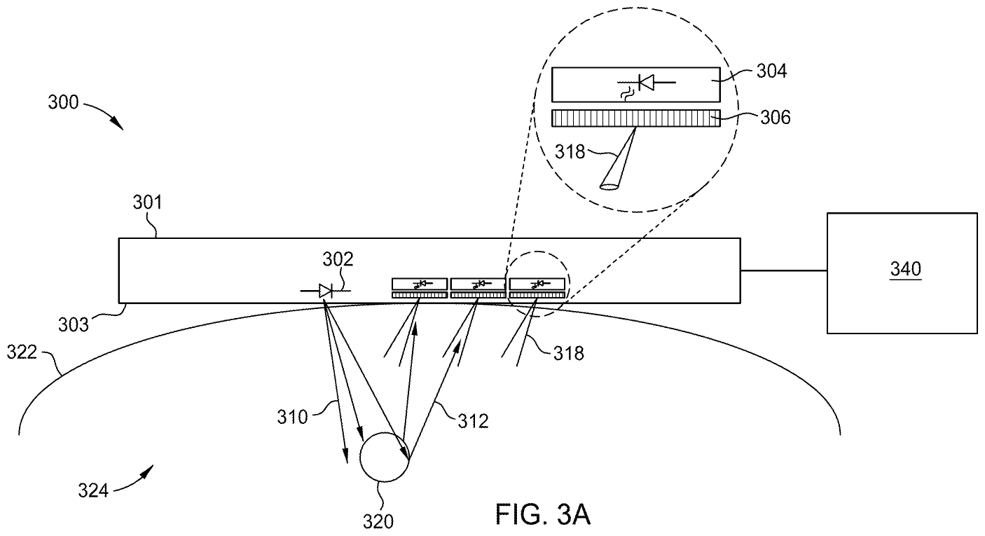

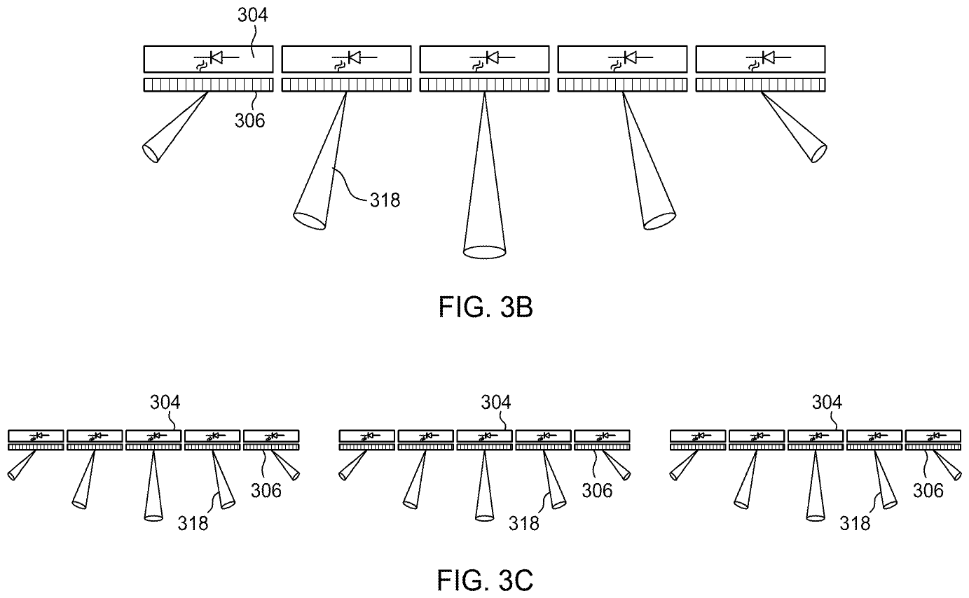

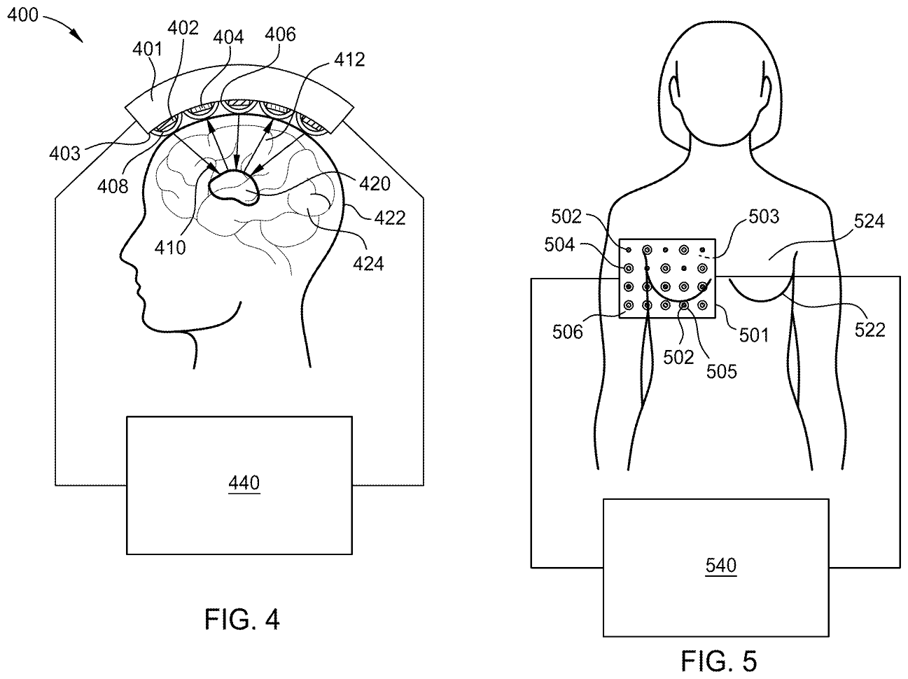

FIGS. 1A-1G illustrate various views of a diffuse optical tomography device (DOT) 100, according to embodiments described herein. FIG. 1A is a cross-sectional view of the DOT device 100 with an interspersed array arrangement 100A of sources and detectors, according to embodiments described herein. FIG. 1B illustrates a view of a surface of a substrate 101 of the DOT device 100 having a rectangular surface configuration 101B. The rectangular surface configuration 101B may be flat, curved or conformal. FIG. 1C illustrates a view of a surface of a substrate 101 of the DOT device 100 having to an annular configuration 101C. FIG. 1D illustrates a cross-sectional view of the DOT device 100 with separate detector and source (SDS) array arrangement 100D. The DOT device 100 described herein includes one of the interspersed array arrangement 100A or the SDS array arrangement 100D and one of the rectangular surface configuration 101B or the annular configuration 101C.

The DOT device 100 includes sources 102 and detectors 104 disposed over a second surface 103 the substrate 101. The second surface 103 of the substrate 101 faces the tissue. The interspersed array arrangement 100A includes the sources 102 and the detectors 104 in an intermingled or an alternating pattern. The SDS array arrangement 100D includes the sources 102 and the detectors 104 as separate arrays.

As shown in FIGS. 1A and 1D, the sources 102 are configured to produce radiation 110. The radiation 110 may be near infrared (NIR) radiation or visible light. The radiation has wavelengths of 500 nm to 2500 nm. More particularly, 600 nm to 1200 nm, or 650 nm to 1000 nm. The source 102 may include infrared (IR) radiation or visible light emitting diodes (LEDs or micro-LEDs), vertical cavity surface-emitting lasers (VCSELs), or other lasers. In FIGS. 1A and 1D, only one ray of radiation 110 from each source 102 is shown for illustrative purposes. In some embodiments, sources 102 are configured to produce radiation 110 at an angle from about 1° to about 179°. Sources 102 may be configured to produce radiation 110 in a range from 500 nm to 2500 nm. Or more particularly, 600 nm to 1200 nm, or 650 nm to 1000 nm. Different ranges may be utilized for different implementations. For example, biological tissue is relatively transparent to radiation in the 700 nm to 900 nm range (e.g., NIR radiation), allowing for penetration into biological tissue. In one instance, a range of 650 nm to 750 nm may be utilized to differentiate oxygenated and de-oxygenated blood. Oxy- and deoxy-hemoglobin are strongly linked to blood and tissue oxygenation and metabolism. In another embodiment, a range of 900 nm to 950 nm may be utilized for detecting lipids. In yet another embodiment, a range of 950 nm to 1000 nm may be utilized to detect water.

As the radiation from the sources 102 is produced and directed toward the target (e.g., a body part or other type of tissue), the target may absorb or scatter the radiation through interaction with the materials, such as tissue and fluids (e.g., oxygenated/deoxygenated blood, lipids, water, etc.). The differences in the absorption and scattering of the radiation detected by the detectors 104 allowed the DOT device to differentiate between the tissue types. For example, differentiation between oxygenated and deoxygenated hemoglobin may assist in the determination of anatomical and functional characteristics of the tissue. In some examples, different absorption spectra of the oxygenated and deoxygenated hemoglobin enables spectroscopic separation of the materials using the emitted radiation to identify tissue. The sources 102 may be coupled together directly, via optical fiber, or by other means. In one embodiment, there is no optical fiber, as sources are mounted against the tissue. Mounting the sources on a substrate surface and placing it against the tissue enables handheld and endoscopic applications. Removing fiber cables further enable simplification, miniaturization, and improved signal from the structures.

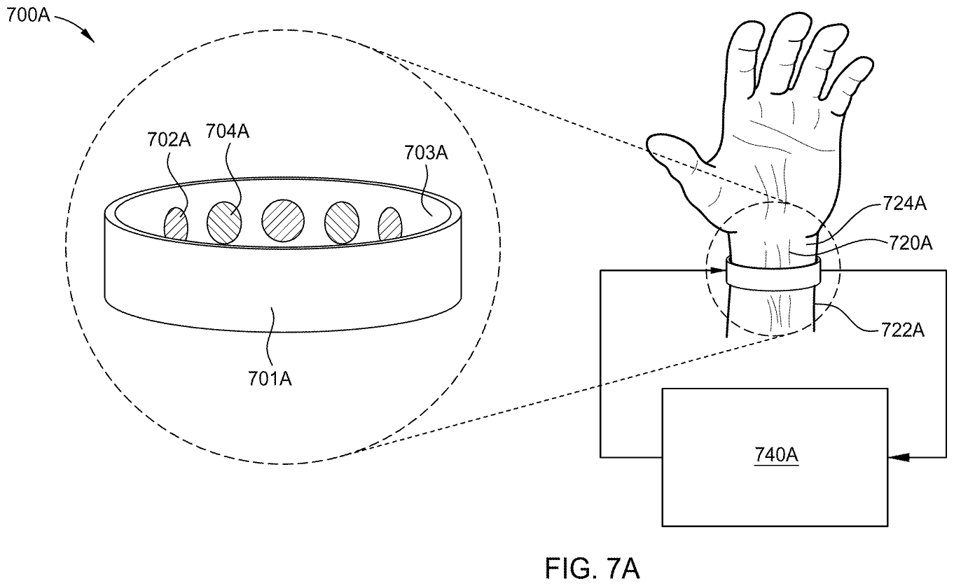

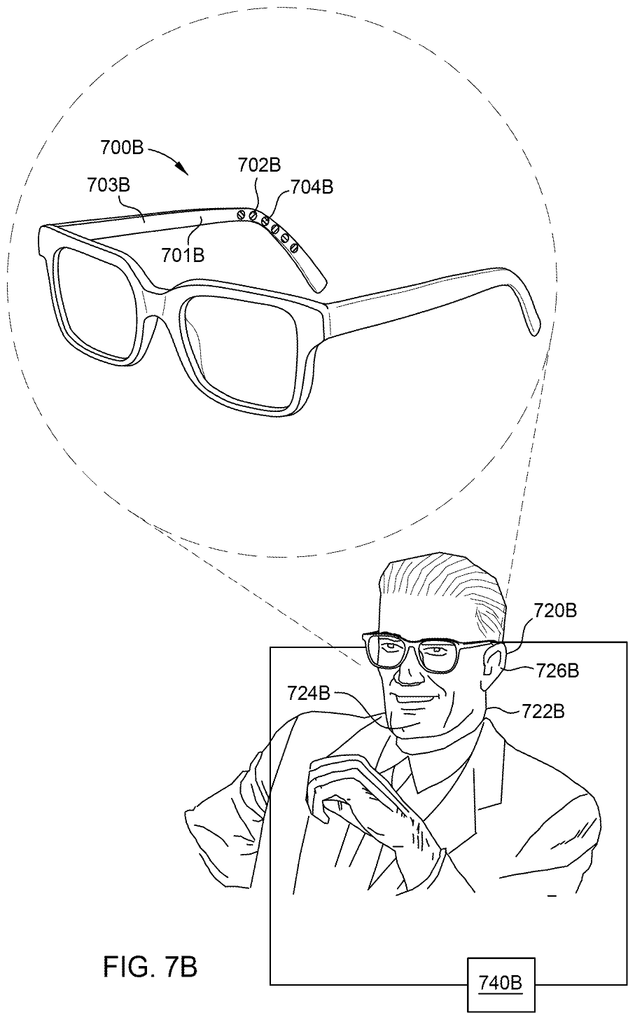

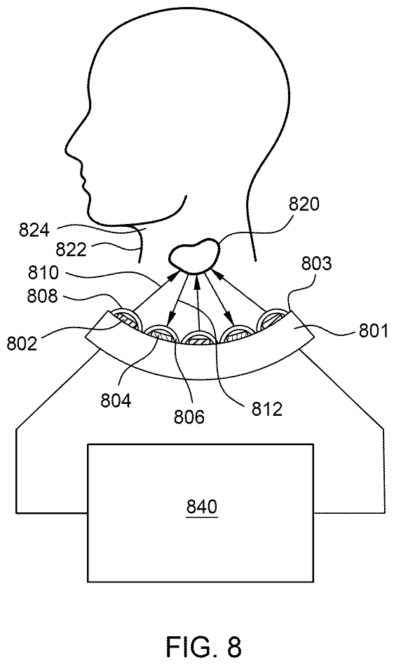

As shown in FIGS. 1A and 1D, the detectors 104 are configured to detect scattered radiation 112. In FIGS. 1A and 1D, only one ray of scattered radiation 112 is shown going to each detector 104 for illustrative purposes. The detectors 104 may be configured to detect scattered radiation 112 produced by the sources 102. The detectors may capture the intensity of the radiation at multiple locations. As shown in further detail in FIG. 3, each detector 104 may be configured to only detect scattered radiation 112 from specified angles, i.e., the detectors are angularly selective. The detectors 104 may be coupled together directly or by other means. In one embodiment, there is no optical fiber, as detectors are mounted on the surface of a substrate that can be held against the tissue. This approach enables handheld and endoscopic applications. Designs without fiber cables further enable simplification, miniaturization, and improved signal from detected scattered radiation.

In FIG. 1D, the sources 102 are clustered in a source array 102D and the detectors 104 are clustered in a detector array 104D. In FIG. 1D, the source array 102D is on the same substrate as the detector array 104D. In other embodiments, the source array 102D may be on a substrate 101 and the detector array 104D may be on a second substrate. As shown in FIG. 1D, the source array 102D is located a distance x away from the detector array 104D.

Within the source array 102D, the sources 102 may be arranged in any repeating or non-repeating design. Within the detector array 104D, the detectors 104 may be arranged in any repeating or non-repeating design.

As shown in FIGS. 1A-1C, the sources 102 are regularly spaced with the detectors 104. In some embodiments, the sources 102 may be irregularly spaced with the detectors 104. In other embodiments, the sources 102 and the detectors 104 may be regularly patterned in any repeating or non-repeating design. Higher source 102 and detector 104 density may be utilized to obtain signals with improved signal to noise ratios. The ability to change the source and detector patterns in space enables different angle filters, which in turn enables high density patterning.

In FIGS. 1A-1C, the sources 102 and the detectors 104 are all located on the same substrate 101. In other embodiments, a plurality of substrates 101 may be utilized. In embodiments with multiple substrates 101, all of the substrates 101 may conform to the interspersed array arrangement 100A or the SDS array arrangement 100D. In other embodiments, the substrates 101 may utilize both the interspersed array arrangement 100A and the SDS array arrangement 100D.

In some embodiments, the substrate 101 may be opaque or transparent to improve the imaging. In embodiments in which the substrate 101 is transparent, the DOT device 100 may include sources 102 and detectors 104 disposed over a first surface 105 the substrate 101. The second surface 103 of the substrate 101 faces the tissue volume 124. The radiation is produced and directed toward the target (e.g., a body part or other type of tissue) by propagating through the substrate 101. In some embodiments, a thinner substrate 101 may be utilized to make the DOT device 100 more portable. In one or more embodiments, substrate 101 may be flexible.

As shown in FIG. 1B, the substrate 101 has a rectangular surface configuration 101B. In FIG. 1C, the substrate 101 has an annular configuration 101C. In other embodiments, the substrate 101 may be any regular or irregular shape.

In FIGS. 1A-1D, the sources 102 and detectors 104 cover the entire area of the second surface 103 of the substrate 101. However, in other embodiments, the sources 102 and detectors 104 may cover a fraction of the surface area of the substrate 101. Although FIGS. 1A-1D show sources 102 and detectors 104 on the second surface 103 of the substrate 101, in other embodiments, the sources 102 and detectors 104 may be present on other surfaces of the substrate 101. In some embodiments, sources 102 and detectors 104 may cover the entire surface area of the substrate 101.

In FIGS. 1A-1D, the plurality of detectors 104 have structures 106 covering the exposed surfaces of the detectors 104. The structures 106 may be configured to be wavelength filters, angular filters, flat lenses, or other types of filters and may perform functions such as edge detection or shape detection. As shown in FIGS. 1A and 1D, the structures 106 may be structures disposed around the detectors 104. In some embodiments, a single structure 106 may cover multiple detectors 104. The structures 106 are designed to selectively filter by angle, wavelength, polarization, or other qualities of emitted or detected radiation. The structures cause diffraction, refraction, or filtering of the radiation entering the detector. The structures may be configured to be wavelength filters, angular filters, flat lenses, or other types of filters and may perform functions such as edge detection or shape detection. The structures can include nanoantennas nanoantennas, nanopillars, nanorods, nanoslitsn, nanoholes, nanowires, nanodisks, nanoislands, or the like extending from or inside the surface of a substrate and may form metalenses, diffractive gratings, or diffractive lenses. In some embodiments, the metalenses are flat lenses. Metalenses, diffractive gratings, or diffractive lenses may be known as metasurfaces. The structures have a height that is related to the wavelength of the radiation emitted. In some embodiments, the height structures may be from about ⅓ of the wavelength emitted to about 3 times the wavelength emitted. In embodiments in which the substrate is transparent, the DOT device 100 may include sources 102 and detectors 104 disposed over of on either side of the substrate. In embodiments in which the substrate is transparent, the structures are disposed over of on either side of the substrate.

In FIG. 1A, the sources 102 have structures 108 covering the exposed surfaces of the sources 102. In FIG. 1D, there are no structures 108 on the sources 102. FIGS. 1B and 1C are divided in half, where one half of each substrate 101 includes sources 102 without structures 108, and one half of each substrate 101 includes sources 102 with structures 108. In other embodiments, any percentage of the sources 102 may be covered by structures 108. As shown in FIG. 1A, the structures 108 may be structures disposed around the sources 102. In some embodiments, a single structure 108 may cover multiple sources 102. In embodiments where more than one source 102 is covered by a structures 108, the sources 102 covered by structures 108 may be arranged in any repeating or non-repeating pattern amongst the uncovered sources 102.

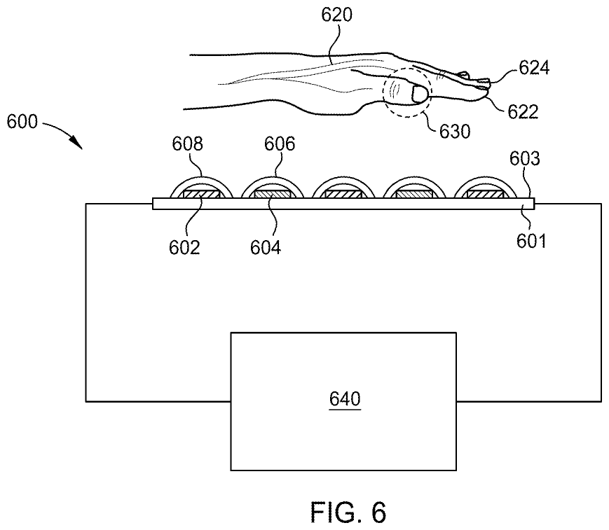

As shown in FIGS. 1A and 1D, the DOT device 100 is in communication with the controller 140. The controller 140 facilitates the control and automation of the method 200 from FIG. 2 for creating a display of the sub-surface feature 120 described herein. The controller 140 may include a central processing unit (CPU), one or more graphics processing units (GPUs), a memory, and support circuits. The CPU may be one of any form of computer processors that are used in industrial settings for controlling various processes and hardware (e.g., motors and other hardware) and monitoring the processes (e.g., the amount of sources 102 emitting radiation 110 and the amount of detectors 104 detecting scattered radiation 112). The memory is connected to the CPU and may be readily available memory, such as random access memory (RAM). Software instructions and data can be coded and stored within the memory for instructing the CPU. The support circuits are also connected to the CPU for supporting the processor. The support circuits may include cache, power supplies, clock circuits, input/output circuitry, subsystems, and the like. A program (or computer instructions) readable by the controller determines which tasks are performable on the DOT device 100. The program may be software readable by the controller 140 and may include code to monitor, for example, the amount of sources 102 emitting radiation 110 and the amount of detectors 104 detecting scattered radiation 112 in a DOT device 100 throughout method 200.

The controller 140 is configured to facilitate the operation of the DOT device 100. In some embodiments, the controller 140 includes one or more inputs (e.g., 3 inputs) for each of the plurality of sources 102 and each of the plurality of detectors 104. In some embodiments, the controller includes one or more inputs with or without a common ground. The controller 140 is operable to select the wavelength of radiation that is emitted from the sources 102. E.g., the controller 140 is operable to select between broad band LED sources configured to emit specific wavelengths based on filters. In some embodiments, the controller 140 may instruct the sources 102 to emit radiation 110 from different wavelengths simultaneously. In some embodiments, the controller 140 may instruct the sources 102 to alternate between or sweep through different wavelengths of radiation 110 by sequentially selecting sources with different wavelengths, or with broadband sources combined with filters to tune the radiation to different wavelengths.

The controller 140 is operable to receive the data of the scattered radiation 112 from the detectors 104. The controller 140 is also operable to create a model predicting the distribution of detected radiation based on one or more models of candidate scattering structures (e.g., veins carrying blood). The controller 140 is further operable to compare the modeled array with the measured data. Based on the comparison, the controller 140 is operable to construct an image based on the modeled and collected data.

In operation, as shown in FIGS. 1A and 1D, the DOT device 100 is applied to a tissue volume 124 such that the sources 102 and detectors 104 are touching the tissue surface 122 of the tissue volume 124. In other embodiments, the sources 102 and detectors 104 may be facing, but not touching, the tissue surface 122. The surface 122 may be skin, hair, internal organs, or other surfaces. Below the surface 122 is a sub-surface feature 120. The sub-surface feature 120 may be a vein, tumor, oxygenated or deoxygenated blood flow, blood clot, or other feature of interest.

The sources 102 emit radiation 110 that passes through the tissue surface 122 and into the tissue volume 124. Some of the radiation 110 contacts the sub-surface feature 120, and becomes scattered radiation 112. Some of the scattered radiation 112 is detected by detectors 104. The detectors 104 transmit the signal of the scattered radiation 112 to the controller 140.

FIG. 1E illustrates a view of a surface of a DOT device 100 with a latticed array arrangement 100E. In FIG. 1E, the sources 102 are regularly spaced and surrounded by a plurality of unit cell detectors 104E to create a unit cell 116. The four unit cell detectors 104E may be equivalent to a single detector 104 when combined, e.g., may have the same area, signal, sensitivity, etc. The unit cell detectors 104E may each be coupled to a filter that operates at selected wavelengths. The filters may made using structures. The structures are designed to selectively filter by angle, wavelength, polarization, or other qualities of emitted or detected radiation. The structures cause diffraction, refraction, or filtering of the radiation entering the detector. The structures may be configured to be wavelength filters, angular filters, flat lenses, or other types of filters and may perform functions such as edge detection or shape detection. The structures include nanoantennas, nanopillars, nanorods, nanoslitsn, nanoholes, nanowires, nanodisks, nanoislands, or the like extending from or inside the surface of a substrate and may form metalenses, diffractive gratings, or diffractive lenses. In some embodiments, the metalenses are flat lenses. Metalenses, diffractive gratings, or diffractive lenses may be known as metasurfaces. The structures have a height that is related to the wavelength of the radiation emitted. In some embodiments, the height structures may be from about ⅓ of the wavelength emitted to about 3 times the wavelength emitted. In embodiments in which the substrate is transparent, the DOT device 100 may include sources 102 and detectors 104 disposed over of on either side of the substrate. In embodiments in which the substrate is transparent, the structures are disposed over of on either side of the substrate.

In FIG. 1E, the unit cells 116 are arranged in a regular array. In other embodiments, there may be spacing between the each of the unit cells 116. In some embodiments, the unit cells 116 may be in an offset pattern, or in any repeating or non-repeating design. In FIG. 1E, the unit cell detectors 104E are shown as having equivalent sizes and shapes. In other embodiments, some unit cell detectors 104E may be larger or smaller than other unit cell detectors 104E. In some embodiments, each unit cell detectors 104E may be any regular or irregular shape.

FIG. 1F illustrates a view of a structure 106F of a first configuration. The structure 106F of the first configuration is a diffractive grating or diffractive lens. The structure 106F includes a structure substrate 150F, a plurality of optical structures 152F, a film stack 154, and a detector 104F. The film stack 154 may include a first film layer 154A and a second film layer 154B. In some embodiments, the film stack 154 may include additional layers configured as an interference or other filter structure. The structure 106F is configured to act as an angular filter for transmitting the radiation at an angle within a certain acceptance cone (as will be described in further detail below). Radiation that is not within the acceptance cone will be either reflected or absorbed by the structure substrate 150F. The optical structures 152F have a pitch and orientation such that when radiation is incident from a particular direction, it is diffracted to be normal to the film stack 154. The acceptance angle can be changed by designing the pitch and the orientation of the diffractive optical structures 152F. The detector 104F detects the radiation that is filtered through the optical structures 152F and the film stack 154.

FIG. 1G illustrates a view of a structure 106G of a second configuration. The structure 106G of the second configuration is a lens, e.g. a metalens. The structure 106G includes a structure substrate 150G, a plurality of grating lines or elements 152G, and a detector 104G. The structure 106G passes the angular power spectrum of the incident radiation using a large field of vision (FOV) lens and the detector 104G. The use of angular selectivity increases the signal to noise ratio at the detector 104G, thus providing more information about the radiation exiting the target (i.e., the tissue).

FIG. 2 is a flow diagram of a method 200 of utilizing a DOT device 100, according to embodiments.

In operation 210, one or more of the sources 102 emit radiation 110. The sources 102 may continuously emit radiation 110. In some embodiments, the sources 102 may emit radiation 110 in a patterned or intermittent fashion. The radiation 110 is emitted as pulsed radiation or a modulated NIR radiation to improve the signal to noise ratio in the detected signal. The radiation 110 may be near infrared (NIR) radiation or visible light. The radiation has wavelengths of 500 nm to 2500 nm. More particularly, 600 nm to 1200 nm, or 650 nm to 1000 nm.

During operation 210, an operator may manually control (or the CPU may control) which sources 102 emit radiation 110, or the number of sources 102 may be programmed into the controller 140. In some embodiments, all of the sources 102 emit radiation 110. In some embodiments, during operation 210, the sources 102 may emit radiation 110 at a constant wavelength. In other embodiments, the sources 102 may emit radiation 110 at two or more wavelengths. Operation 210 may be performed concurrently with operation 220, operation 230, and/or operation 240.

In operation 215, the radiation is scattered by a sub-surface feature 120. As shown in FIGS. 1A and 1D, the sub-surface feature 120 scatters the radiation 110 into scattered radiation 112. During operation 210, methods of reducing the radiation 110 that does not interact with the sub-surface feature 120 may be employed to improve signal noise and reduce the computational burden on the controller 140 during operation 230. For example, the tissue volume 124 scatters the radiation 110 into noise (not shown). When the noise is detected by the detectors in operation 220, the noise with no useful information content is added to the detector signal generated by the detectors 104 without improving the clarity of the generated image. When the sources 102 are covered by structures 106, radiation 110 at different wavelengths may be emitted from the sources 102. When more targeted wavelengths are utilized, the noise to signal ratio is improved. The improved signal to noise ratio increases data/information content in the signal collected by the detectors 104. The improved signal to noise ratio in the signal collected by the detectors 104 reduces the computational burden on the controller 140 in operation 230, thus improving the data or image quality or reducing the acquisition time for a given result.

In operation 220, one or more of the detectors 104 detect the scattered radiation 112. During operation 220, methods of improving the signal collected by the detectors 104 may be employed to reduce the computational burden on the controller 140 during operation 230. When the detectors 104 have structures 106, the angles and/or wavelengths of the detectable scattered radiation 112 is reduced, improving the signal to noise ratio in the signal collected by the detectors 104. The number of detectors 104 collecting scattered radiation 112 designed into a particular DOT system may be increased or decreased based on the amount, angle, etc. of scattered radiation 112 collected during operation 220. When fewer detectors 104 are configured to collect scattered radiation, less signal is collected. These methods of improving signal collected by the detectors 104 may also be used to reduce the computational burden on the controller 140 in operation 230.

Operation 220 may be performed concurrently with operation 210. Operation 220 may be performed concurrently with operation 230 and/or operation 240.

In operation 230, the signal from the detectors 104 is processed to produce data and minimize errors from noise. In some embodiments, an image of the sub-surface feature 120 is generated. In some embodiments, the image of the sub-surface feature 120 is generated by solving the reverse scattering problem. The reverse scattering problem utilizes one or more models and/or one or more algorithms. Solving reverse scattering problem estimates the distribution of optical parameters (such as absorption and scattering coefficient) inside a tissue (or other body part or target) based on measurements of radiation scattering.

In some embodiments, operation 230 is performed using a precomputed result. The signals collected by the detectors 104 are compared with one or more precomputed results. If the data collected by the detectors 104 reasonably matches the precomputed result, a positive match is reported. If the data collected by the detectors 104 does not substantially match the precomputed result, a negative match is reported. The precomputed results may be stored in a lookup table. In embodiments utilizing a precomputed result, an image may not be generated, and operation 240 would be replaced with the precomputed results. A precomputed result may be utilized, for example, to uniquely identify a user based on their blood vessel signature.

Operation 230 may be performed in the controller 140. A neural network may be utilized to implement and optimize operation 230. Operation 230 may be performed concurrently with operation 210 and/or operation 220. Operation 220 may be performed concurrently with operation 240.

In operation 240, an image or other data is displayed. The image may be displayed on a monitor, projector, TV, or other method of presenting images. The size and image quality of the display is dependent on the amount of data collected by the detectors 104 that is directed to the sub-surface feature 120. Operation 240 may be performed concurrently with or after operation 210, operation 220, and/or operation 230.

Each of the operations described herein may occur simultaneously. In some embodiments, the signal may be stored and the computation may be performed at a later time.

FIG. 3A illustrates a schematic cross-sectional view of a DOT device 300 with angularly selective structures 306. FIG. 3B illustrates a schematic cross-sectional view of an array of detectors 304 with a range of angularly selective structures 306. FIG. 3C illustrates a schematic cross-sectional view of multiple arrays of a detectors 304 with repeating groups of angularly selective structures.

The DOT device 300 may be the DOT device 100 of FIGS. 1A-1E. As such, the substrate 301 may be the substrate 101, the second surface 303 may be the second surface 103, the sources 302 may be the sources 102, the detectors 304 may be the detectors 104, the structures 306 may be the structures 106, the radiation 310 may be the radiation 110, the scattered radiation 312 may be the scattered radiation 112, the sub-surface feature 320 may be the sub-surface feature 120, the surface 322 may be the surface 122, the tissue volume 324 may be the tissue volume 124, and the controller 340 may be the controller 140. Features of the DOT device 100 of FIGS. 1A-1E may be combined with the features of the DOT device 300 of FIG. 3.

In FIG. 3A, a single source 302 emits radiation 310 through a surface 322 into a tissue volume 324. The radiation 310 may be near infrared (NIR) radiation or visible light. The radiation has wavelengths of 500 nm to 2500 nm. More particularly, 600 nm to 1200 nm, or 650 nm to 1000 nm. The source 302 may be one or a plurality of source 302. Some of the radiation 310 interacts with a sub-surface feature 320 that scatters the radiation to produce scattered radiation 312. The detectors 304 are arranged so that the scattered radiation passes through a structure 306 prior to being detected by the detectors 304. As shown in FIG. 3, the structures 306 limits the scattered radiation 312 that can be received by the detector 304 to a specified angular cone 318. When the scattered radiation 312 is angled towards a detector 304 within the cone 318, the detector 304 is able to detect the scattered radiation 312. When the path of the scattered radiation 312 is towards a detector 304, but not within the cone 318, the scattered radiation 312 is not detected by the detector 304, thus reducing the amount of noise in the detector signal and improving the signal to noise ratio. The signal of the received scattered radiation 312 is transmitted to the controller 340.

A structure 306 may be tuned to have an angular radiation acceptance cone 318 with a detection angle ranging from about 1° to about 179°. Or more precisely, the detection angle of cone 318 may range from about 45° to about 135°, such as about 5° to about 10°, such as about 10° to about 20°, such as about 20° to about 50°. The structures 306 may be tuned such that the cone 318 detects scattered radiation 312 at angles ranging from about 1° to about 179°, wherein the angles are formed between the cone 318 and the structures 306. The structure angular acceptance cone 318 may be, but is not limited to, a cone shape, and may include an elongated cone shape or other shapes.

In FIG. 3A, the structures 306 are shown to have a cone 318 of detection. In other embodiments, the structures 306 may be configured to only detect scattered radiation 312 at certain wavelengths. In some embodiments, the structures 306 may be configured to both have a cone 318 of detection and only detect scattered radiation 312 at certain wavelengths or polarizations.

In some embodiments, structures 306 are only shown on the detectors 304 in FIG. 3. In FIG. 3, the sources 302 do not have structures and are capable of emitting radiation 310 at a variety of angles. In some embodiments, the sources 302 may have structures which restrict the angle, wavelength, polarization, or combination thereof of radiation 310.

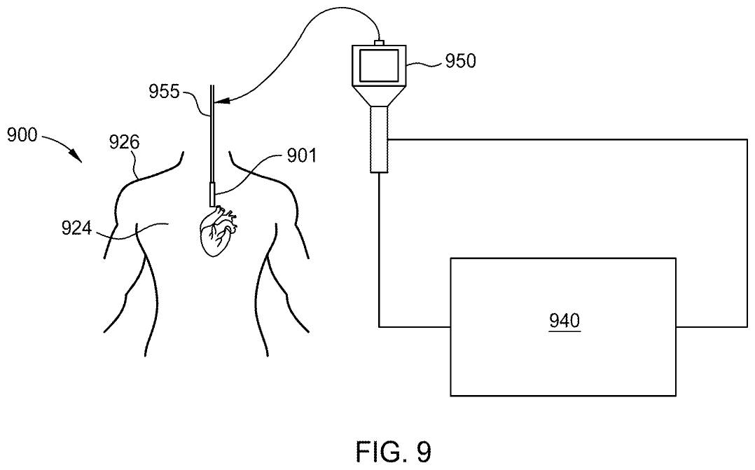

FIGS. 4-9 illustrate exemplary uses of a DOT device, according to embodiments described herein. The uses shown in FIGS. 4-9 are non-exhaustive.

FIG. 4 illustrates an exemplary use of a DOT device 400, according to embodiments. The DOT device 400 may be used in place of the DOT device 100 of FIGS. 1A-1E. Features of the DOT device 100 of FIGS. 1A-1E and/or DOT device 300 of FIG. 3 may be combined with the features of the DOT device 400 of FIG. 4.

In FIG. 4, the DOT device 400 has a substrate 401 that is curved, which conforms to the head 422. The sources 402 and the detectors 404 cover the entirety of the second surface 403 of the substrate 401. The sources 402, covered by structures 408, emit radiation 410 through the head 422 and into the cranial material 424. The radiation 410 may be near infrared (NIR) radiation or visible light. The radiation includes light in the range of 500 nm to 2500 nm. More particularly, 600 nm to 1200 nm, or 650 nm to 1000 nm. The radiation 410 that interacts with the sub-surface feature 420 becomes scattered radiation 412. The detectors 404, covered by structures 406, detect the scattered radiation 412. The data of the detected scattered radiation 412 is transmitted to the controller 440.

In FIG. 4, the DOT device 400 is applied to a head 422 to capture a DOT signal of the cranial material 424. The purpose of the cranial material 424 DOT signal may be to monitor brain activity, scan for a tumor, scan for a blood clot, monitor blood oxygenation, monitor metabolism, or monitor for an existing or potential stroke, aneurism, or other neurological disease. In some embodiments, functional NIR spectroscopy (fNIRS) may be used to monitor the changes in oxygenation and blood flow of the brain. The sub-surface feature 420 may be a blood vessel, a tumor, or other feature within the brain. In some embodiments, the sources 402 and detectors 404 may have structures 406, 408 selected to detect a neurological environment. In some embodiments, the sources 402 and detectors 404 may have structures 406, 408 selected to detect wavelengths from a specific neurological study being performed.

In some embodiments, the substrate 401 may be flexible to conform to the contours of the head 422. In some embodiments, the substrate 401 may have a curvature based on the curvature of the average human head 422. In some embodiments, the substrate 401 may include multiple smaller substrates 401 that are attached to an adjustable helmet (not shown). The size of the adjustable helmet may be increased or decreased based on the size of the head 422.

The head 422 may be bald, as shown in FIG. 4. The head 422, may have hair (not shown) between the head 422 and the sources 402 and detectors 404.

FIG. 5 illustrates an exemplary use of a DOT device 500, according to embodiments. The DOT device 500 may be the DOT device 100 of FIGS. 1A-1E. Features of the DOT device 100 of FIGS. 1A-1E, the DOT device 300 of FIG. 3, and/or the DOT device 400 of FIG. 4 may be combined with the features of the DOT device 500 of FIG. 5.

In FIG. 5, the DOT device 500 has a, rectangular substrate, which is applied to the breast surface 522. The sources 502 and the detectors 504 cover the of the second surface 503 of the substrate 501. The sources 502, where some of the sources 502 are covered by structures 508, emit radiation (not shown) through the breast surface 522 and into the breast material 524. The radiation that interacts with sub-surface features (not shown), such as a tumor, a cyst, or other similar features, within the breast material 524 becomes scattered radiation (not shown). The detectors 504, covered by structures 506, detect the scattered radiation. The data of the detected scattered radiation is transmitted to the controller 540. The radiation may be near infrared (NIR) radiation or visible light. The radiation includes light in the range of 500 nm to 2500 nm. More particularly, 600 nm to 1200 nm, or 650 nm to 1000 nm.

In FIG. 5, the DOT device 500 is applied to a breast surface 522 to capture a DOT signal of the breast material 524. The purpose of the breast material 524 DOT signal may be to screen for cancer, monitor tumor growth, monitor angiogenesis, or other diseases of the breast tissue. In some embodiments, the sources 502 and detectors 504 may have structures 506, 508 tuned to a mammographic processing environment.

Although a singular substrate is shown in FIG. 5, multiple substrates 501 may be utilized. Each substrate 501 may be a different shape and may be flexible/wearable. In one embodiment, a first substrate 501 is placed on top of the breast surface 522, and a second substrate 501 is placed on the bottom of the breast surface 522. In this embodiment, either the interspersed array arrangement 100A or the SDS array arrangement 100D may be utilized. In this embodiment, pressure may be applied to the first and second substrates 501 to decrease the amount of breast material 524 that is covered by each source 502 and detector 504. In some embodiments, pressure may not be applied to the first and second substrates 501.

FIG. 6 illustrates an exemplary use of a DOT device 600, according to embodiments. The DOT device 600 may be used in place of the DOT device 100 of FIGS. 1A-1E. Features of the DOT device 100 of FIGS. 1A-1E, the DOT device 300 of FIG. 3, the DOT device 400 of FIG. 4, and/or the DOT device 500 of FIG. 5 may be combined with the features of the DOT device 600 of FIG. 6.

In FIG. 6, the DOT device 600 has a flat, rectangular substrate, and a hand 622 may be applied to the DOT device 600. The sources 602 and the detectors 604 cover the entirety of the first surface 603 of the substrate 601. The sources 602, covered by structures 608, emit radiation (not shown) through the surface of the hand 622 and into the hand subcutaneous tissue 624. Source wavelengths may be selected for oxygenated blood, deoxygenated blood, or other type of bodily fluid or tissue. The radiation that interacts with structure of the tissue 620 becomes scattered radiation (not shown). The detectors 604, covered by structures 606, detect the scattered radiation. The signal of the detected scattered radiation is transmitted to the controller 640. The radiation may be near infrared (NIR) radiation or visible light. The radiation includes light in the range of 500 nm to 2500 nm. More particularly, 600 nm to 1200 nm, or 650 nm to 1000 nm.

In FIG. 6, the hand 622 is applied to the DOT device 600 to perform vein mapping. Vein mapping acquires the pattern of the veins within the hand 622. In some embodiments, a user may apply a finger 630 to the DOT device 600. The DOT device 600 may recognize the spatio-spectral signature of the blood vessels (not shown) within the finger 630. The user may be identified based on the signature of the blood vessels. The blood vessel or vein pattern on the hand 622 or the blood vessel signature can be utilized for authentication of personnel for computer, phone, building, vehicular, or other forms of access. Other use cases for blood vessels includes venipuncture (injection) or robotic surgery.

FIG. 7A illustrates an exemplary use of a DOT device 700A, according to embodiments. The DOT device 700A may be used in place of the DOT device 100 of FIGS. 1A-1E. Features of the DOT device 100 of FIGS. 1A-1E, the DOT device 300 of FIG. 3, the DOT device 400 of FIG. 4, the DOT device 500 of FIG. 5, and/or the DOT device 600 of FIG. 6 may be combined with the features of the DOT device 700 of FIG. 7A.

In FIG. 7A, the DOT device 700A has a ring substrate, which is curved around a user's arm 722A. The sources 702A and the detectors 704A cover a portion of the interior surface 703A of the substrate 701A. The sources 702A emit radiation (not shown) through the skin of the arm 722A and into the arm subcutaneous tissue 724A. The radiation that interacts with structures such as blood vessels, veins 720A, etc. is scattered and becomes scattered radiation (not shown). The detectors 704A, covered by structures (not shown), detect the scattered radiation. The signal of the detected scattered radiation is transmitted to the controller 740A. The radiation may be near infrared (NIR) radiation or visible light. The radiation includes light in the range of 500 nm to 2500 nm. More particularly, 600 nm to 1200 nm, or 650 nm to 1000 nm.

In FIG. 7A, the DOT device 700A is applied to the arm 722A to monitor blood flow in the veins 720A. The blood flow in the veins 720A can be utilized to track heart rate, blood oxygen concentration, and/or sleep patterns for fitness trackers, smart watches, medical equipment, and other heart rate monitors. The DOT device 700A may also be utilized to visualize veins 720A.

Although DOT device 700A is shown around an arm 722A in FIG. 7A, the DOT device 700A may be sized to fit around a chest, leg, finger, neck, or other body part to measure heart rate, blood oxygenation, and other parameters.

The DOT device 700A may be further configured to store heart rate data. The DOT device 700A may also include accessories to be Bluetooth compatible. The controller 740A may include two or more sub-controllers. For example, a first controller may be incorporated into the substrate 701A to control the source 702A and detector 704A operations. A second controller, such as an application on a phone or computer, may be configured to process the data collected by the detectors 704A.

FIG. 7B illustrates an exemplary use of a DOT device 700B, according to embodiments. The DOT device 700B may be used in place of the DOT device 100 of FIGS. 1A-1E. Features of the DOT device 100 of FIGS. 1A-1E, the DOT device 300 of FIG. 3, the DOT device 400 of FIG. 4, the DOT device 500 of FIG. 5, the DOT device 600 of FIG. 6, and/or the DOT device 700A of FIG. 7A may be combined with the features of the DOT device 700 of FIG. 7A.

In FIG. 7B, the DOT device 700B has a pair of glasses 701B, which are placed on a user's head 722B. The sources 702B and the detectors 704B cover a portion of the interior surface 703B of the glasses 701B. The sources 702B emit radiation (not shown) through the surface of the head 722B and into the head subcutaneous tissue 724B. The radiation that interacts with blood vessels 720B becomes scattered radiation (not shown). The detectors 704B, covered by structures (not shown), detect the scattered radiation. The signal of the detected scattered radiation is transmitted to the controller 740B.

In some embodiments, the DOT device 700B may have a single source 702B. In some embodiments, this single source 702B may utilize laser imaging, detection, and ranging (LIDAR) technology. In other embodiments, the DOT device 700B may include multiple sources 702B. In some embodiments, the DOT device 700B may be designed with an array of multiple (e.g., fifteen) sources 702B.

In FIG. 7B, the DOT device 700B is applied to the head 722B to monitor blood flow in the blood vessels 720B. In some embodiments, the DOT device 700B may be utilized to recognize the pattern of the user's sub-surface blood vessels 720B which come into view as the glasses are slid over the user's ears 726B. The DOT device 700B may be configured to recognize each blood vessel's 720B characteristic spatial pattern of sub-surface scattered radiation based on the blood vessel's 720B depth, geometry, and size. In some embodiments, the DOT device 700B may also be configured to enable brain computer interfaces. The brain computer interfaces may be configured for use in smart glasses, augmented reality (AR) content, or other uses. In some embodiments, the DOT device 700B may be configured to be utilized in health monitoring applications.

FIG. 8 illustrates an exemplary use of a DOT device 800, according to embodiments. The DOT device 800 may be the DOT device 100 of FIGS. 1A-1E. As such, the substrate 801 may be the substrate 101, the interior surface 803 may be the second surface 103, the sources 802 may be the sources 102, the detectors 804 may be the detectors 104, the structures 806 may be the structures 106, the structures 808 may be the structures 108, the radiation 810 may be the radiation 110, the scattered radiation 812 may be the scattered radiation 112, the sub-surface feature 820 may be the sub-surface feature 120, the neck 822 may be the surface 122, the neck subcutaneous tissue 824 may be the tissue volume 124, and the controller 840 may be the controller 140. Features of the DOT device 100 of FIGS. 1A-1E, the DOT device 300 of FIG. 3, the DOT device 400 of FIG. 4, the DOT device 500 of FIG. 5, the DOT device 600 of FIG. 6, and/or the DOT device 700A, 700B of FIGS. 7A-7B may be combined with the features of the DOT device 800 of FIG. 8.

In FIG. 8, the DOT device 800 has a substrate 801, which conforms to the neck 822. The sources 802 and the detectors 804 cover the interior surface 803 of the substrate 401. The sources 802, covered by structures 808, emit radiation 810 through the neck 822 and into the neck subcutaneous tissue 824. The radiation 810 that interacts with the sub-surface feature 820 is scattered and becomes scattered radiation 812. The detectors 804, covered by structures 806, detect the scattered radiation 812. The signal of the detected scattered radiation 812 is transmitted to the controller 840. The radiation 810 may be near infrared (NIR) radiation or visible light. The radiation includes light in the range of 500 nm to 2500 nm. More particularly, 600 nm to 1200 nm, or 650 nm to 1000 nm.

The sub-surface feature 820 may be the carotid artery or the thyroid gland. The carotid artery may be monitored by the DOT device 800 to monitor a patient's risk for stroke. The carotid artery may also be monitored by the DOT device 800 for other diagnostic or monitoring purposes. The thyroid gland may be monitored by the DOT device 800 to diagnose hyperthyroidism or hypothyroidism. The thyroid gland may also be monitored by the DOT device 800 to check thyroid growths for the potential to become cancerous tumors. For example, cancerous tumors will attract increased volumes of blood flow, while benign growths will have much lower volumes of blood flow. The thyroid gland may further be monitored by the DOT device 800 to monitor other thyroidal conditions or diagnose other thyroid diseases.

FIG. 9 illustrates an exemplary use of a DOT device 900, according to embodiments. The DOT device 900 may be the DOT device 100 of FIGS. 1A-1E. As such, the substrate 901 may be the substrate 101, the body cavity 924 may be the tissue volume 124, and the controller 940 may be the controller 140. Features of the DOT device 100 of FIGS. 1A-1E, the DOT device 300 of FIG. 3, the DOT device 400 of FIG. 4, the DOT device 500 of FIG. 5, the DOT device 600 of FIG. 6, the DOT device 700A, 700B of FIGS. 7A-7B, and/or the DOT device 800 of FIG. 8 may be combined with the features of the DOT device 900 of FIG. 9.

In FIG. 9, the DOT device 900 has a substrate 901, which is capable of being inserted into the body 926. The sources (not shown) and the detectors (not shown) may cover a portion of or the entirety of the surface the substrate 901. The sources emit radiation (not shown) through the body cavity 924 towards the feature of interest. The radiation that interacts with the feature of interest becomes scattered radiation (not shown). The detectors, covered by structures (not shown), detect the scattered radiation. The signal of the detected scattered radiation is transmitted to the controller 940. The radiation may be near infrared (NIR) radiation or visible light. The radiation includes light in the range of 500 nm to 2500 nm. More particularly, 600 nm to 1200 nm, or 650 nm to 1000 nm.

In FIG. 9, the DOT device 900 is connected to a remote 950. The remote is connected to the DOT device 900 via a wire 955. The remote 950 is capable of controlling the depth and positioning of the DOT device 900 within the body 926. The DOT device 900 may be inserted into the body 926 via the mouth, nose, ear, incision, or other opening in the body 926. During a scan, the DOT device 900 may be positioned within the body 926 in the esophagus, stomach, colon, or other cavity within the body 926. In some embodiments, a camera (not shown) may be attached to the substrate 901 to aid positioning the DOT device 900 within the body 926.

In some embodiments, the remote 950 may be hand-held. In other embodiments, the remote 950 may incorporated into a personal computer or mobile device (e.g., iPad, iPhone, etc.). The remote 950 may have an interface for an operator to control the movement of the DOT device 900.

In some embodiments, the DOT device 900 may be utilized to probe the heart (transesophageal cardiography), the colon (colonoscopy), the GI tract (esophagogastroduodenoscopy), or other areas of the body 926.

In other embodiments, the DOT device 100 may be utilized to interact with produce (i.e., fruits and vegetables) to determine the quality (e.g., ripeness, rot, other damage, worm or pest damage, or other quality metrics) of the produce. The DOT device emits radiation at the surface of the produce to create an image of the interior of the produce. The radiation may be NIR radiation. The data from the signal of the scattered radiation is compared to a repository of data to identify the produce. Based on the data of the produce, access to one or more of a computer, a building, a room, a dataset, a car, or a phone is allowed or disallowed. The data from the signal of the scattered radiation is compared with the repository of data to calculate and monitor quality. The result is output to a computing device, such as a smart watch, a fitness tracker, a phone, or other application.

In still another embodiment, the DOT device may be utilized to interact with muscles to assess oxygenation of the blood and metabolism. The interaction with the muscles may aid in the study of muscle function and performance.

While the foregoing is directed to embodiments of the present disclosure, other and further embodiments of the disclosure may be devised without departing from the basic scope thereof, and the scope thereof is determined by the claims that follow.

1. A diffuse optical tomography (DOT) device, comprising:
a substrate;
one or more radiation sources disposed over or under a surface of the substrate;
a plurality of detectors wherein each detector of the plurality of detectors has a first surface and a second surface, the first surface opposite the second surface, the first surface of the plurality of detectors disposed over or under the surface of the substrate; and
structures disposed over the second surface of each of the plurality of detectors, wherein the structures cause diffraction, refraction, or filtering of the radiation entering the detectors.

2. The DOT device of claim 1, wherein the one or more radiation sources and the plurality of detectors are arranged in an interspersed array.

3. The DOT device of claim 1, further comprising:
a controller configured to:
control an emission of radiation from the one or more sources; and
receive a plurality of signal indicative of scattered radiation detected by from the plurality of detectors.

4. The DOT device of claim 3, wherein the controller is further configured to:
process the plurality of signals indicative of the scattered radiation into data.

5. The DOT device of claim 3, wherein the controller is further configured to:
solve a reverse scattering problem; and
generate an image or other data visualization.

6. The DOT device of claim 3, wherein the controller is further configured to:
process detected scattered radiation into data;
compare the data derived from the detected scattered radiation with a plurality of stored data in a data repository; and
output a result.

7. The DOT device of claim 1, further comprising:
one or more source structures; and
wherein the one or more sources comprises:
a second surface opposite the first surface, wherein each of the one or more source structures is disposed over a second surface of each of the one or more sources; and
wherein the first surface is disposed over the substrate.

8. The DOT device of claim 1, wherein the structures are configured to selectively receive scattered radiation based on an angle of the scattered radiation.

9. The DOT device of claim 6, wherein an angle of the scattered radiation ranges from 1° to 179°.

10. The DOT device of claim 1, wherein the structures are configured to selectively receive scattered radiation based on a wavelength of the scattered radiation.

11. The DOT device of claim 3, wherein the radiation is near infrared (NIR) radiation or visible light.

12. The DOT device of claim 1, wherein the structures are metalenses, diffractive gratings, or diffractive lenses.

13. The DOT device of claim 12, wherein the metalenses are flat lenses.

14. A diffuse optical tomography (DOT) device comprising:
a source substrate;
a detector substrate;
one or more sources disposed over or under a source surface of the source substrate;
a plurality of detectors wherein each detector of the plurality of detectors has a first surface and a second surface, the first surface opposite the second surface, the first surface of the plurality of detectors disposed over or under a detector surface of the detector substrate; and
a plurality of structures, wherein the plurality of structures are disposed over the second surface of each of the plurality of detectors, wherein the structures cause diffraction, refraction, or filtering of the radiation entering the detectors.

15. The DOT device of claim 14, wherein the structures are metalenses, diffractive gratings, or diffractive lenses.

16. The DOT device of claim 15, wherein the metalenses are flat lenses.

17. The DOT of claim 14, further comprising a controller configured to:
control emission of radiation from the one or more sources; and
detect scattered radiation from the plurality of detectors.

18. The DOT device of claim 17, wherein the controller is further configured to:
translate the detected scattered radiation signal into data;
solve a reverse scattering problem; and
generate an image.

19. The DOT device of claim 17, wherein the radiation is near infrared (NIR) radiation or visible light.

20. The DOT device of claim 18, wherein the controller is further configured to:
translate detected scattered radiation into data;
compare the data of the detected scattered radiation with a plurality of stored data in a data repository; and
output a result.

21. A method of diffuse optical tomography (DOT) comprising:
emitting radiation from one or more sources of a DOT device, wherein the radiation is scattered;
detecting scattered radiation with a plurality of detectors of the DOT device, wherein the plurality of detectors have a plurality of structures disposed thereover, wherein the structures cause diffraction, refraction, or filtering of the radiation entering the detector; and
translating the scattered radiation that is detected into data.

22. The method of claim 21, wherein the structures are metalenses, diffractive gratings, or diffractive lenses.

23. The method of claim 22, wherein the metalenses are flat lenses.

24. The method of DOT of claim 21, further comprising:
solving a reverse scattering problem to create an image; and
displaying the image.

25. The method of DOT of claim 24, wherein the DOT device is configured to be applied to a surface.

26. The method of DOT of claim 25, wherein the surface includes a body part selected from the group consisting of a head, a breast, an abdomen, a lump, a tumor, a heart, or a lung.

27. The method of DOT of claim 26, wherein:
the radiation is directed towards an interior of the body part.

28. The method of DOT of claim 26, further comprising:
comparing the data of the scattered radiation with a repository of data to identify a person; and
allowing or disallowing access to one or more of a computer, a building, a room, a dataset, a car, or a phone based on the data.

29. The method of DOT of claim 26, further comprising:
comparing the data of the scattered radiation with a repository of data to one or more of:
calculate and monitor heart rate;
calculate and monitor blood oxygen levels; or
calculate and monitor sleep patterns; and
outputting a result to a computing device.

30. The method of DOT of claim 29, wherein the computing device comprises a smart watch, a fitness tracker, a phone, a display, or an application.

31. The method of DOT of claim 25, wherein the surface includes a produce.

32. The method of DOT of claim 31, further comprising:
directing the radiation towards an interior of the produce.

33. The method of DOT of claim 32, wherein an image or other data representative of the interior of the produce is created.

34. The method of DOT of claim 33, further comprising:
comparing the data of the scattered radiation with a repository of data to identify the produce.

35. The method of DOT of claim 33, further comprising:
comparing the data of the scattered radiation with a repository of data to calculate and monitor produce quality; and
outputting a result to a computing device.

36. The method of DOT of claim 35, wherein the computing device comprises a smart watch, a fitness tracker, a phone, or an application.

37. The method of DOT of claim 33, wherein:
the radiation is near infrared (NIR) radiation or visible light.
